(19) United States
(12) Patent Application Publication
(10) Pub. No.: US 2011/0217340 A1
(43) Pub. Date: Sep. 8, 2011

(54) NANODISPERSE COMPOSITIONS

(76) Inventors: Doris Angus, Liverpool (GB); Alison Jayne Foster, Liverpool (GB); David Duncalf, Liverpool (GB); Steven Paul Rannard, Liverpool (GB); Dong Wang, Liverpool (GB)

(21) Appl. No.: 13/059,462

(22) PCT Filed: Jul. 24, 2009
(86) PCT No.: PCT/EP2009/059546

§ 371 Date: May 13, 2011

(30) Foreign Application Priority Data
Aug. 18, 2008 (GB) 0814953.6

(51) Int. Cl. A61K 9/14; A61K 38/43; A61K 31/122; C08K 5/23; C08K 3/16; B82Y 30/00

(52) U.S. Cl. 424/400; 424/94.1; 514/690; 524/190; 524/401; 977/773

(57) ABSTRACT

A process for the production of a soluble composition comprising a water-insoluble active which comprises either the steps of: a) providing a liquid mixture comprising: i) a dissolved water-insoluble active, ii) a dissolved water-soluble carrier, iii) a solvent for each of the active and the carrier, and b) spray-granulating the mixture to remove the, or each, solvent and obtain a substantially solvent-free nano-dispersion of the water-insoluble active in the carrier said water-insoluble active being in nano-particles having a size range of 999-20 nm; or, the steps of: a) providing a substantially solvent-free powder comprising a water-insoluble active dispersed in a water soluble carrier, said water-insoluble active being in nano-particles having a size range of 999-20 nm, and, b) spray-granulating the powder to obtain larger and denser particles. The invention also relates to solvent-free, granular products with a particle size in the range 20 microns to 10 mm and a bulk-density of greater than 0.4 g/cm3 comprising a water-soluble carrier material, the carrier material having dispersed therein a water-insoluble active, wherein on addition of water the carrier dissolves to form an aqueous dispersion of the active with a peak (z-average) particle size of below 800 nm, preferably below 500 nm and more preferably below 200 nm. The invention also relates to an analogous “reverse” process in which the active is water soluble and the carrier is oil soluble.

### FIELD OF THE INVENTION

The present invention relates improvements relating to nano-disperse compositions.

### BACKGROUND OF THE INVENTION

Many materials with desirable properties (herein referred to as “actives”) are water-insoluble or have a very low water-solubility. In the case of pharmaceuticals this can, for example, make their administration difficult and their bioavailability low. Similar problems arise with biocides such as insecticides and herbicides. Also, there are many photo-active materials dyestuffs and pigments which exhibit poor water solubility. Approaches to this problem have been manifold and have included grinding and milling materials to form fine particles. However there are practical limits to milling and grinding and it is difficult to obtain materials with a particle size below 1 micron. It is not believed possible to achieve particle sizes below 0.5 micron by milling.

U.S. Pat. No. 4,830,858 discloses lipid-soluble (water insoluble) materials that are delivered by incorporation into the hydrophobic part of the liposomal bi-layer where they exist as individual molecules “dissolved” in the fatty chains of the lipid forming the bilayer. Such liposomes are unstable and '858 proposes that organic solvent should be removed from a solution of liposome components including a liposome forming amphipathic lipid to form a dried preparation which on addition to water will re-create liposomes. Spray-drying is suggested as a means of removing the organic solvent. This approach is in effect a “dried emulsion” where the solvent for the lipid-soluble material is not removed and re-creates the “emulsion” structure (the liposomes) on the addition of water.

US 2004/242427 discloses a powder containing the water-insoluble crop-protection agent “cinidon-ethyl” and a polymer. It is concerned with how the bio-availability of the agent may be increased by providing it in a very fine form. The patent states how conventional grinding processes (milling) cannot reduce the particle size below 0.5 microns (500 nm) and provides a process in which a polymer and the active are dissolved in one or more organic solvents to obtain a solution which is dried (spray drying can be used). Most importantly, the material obtained is characterised by being in an amorphous state as determined by X-ray crystallography. The skilled-worker would understand from the comments about the amorphous state that this is a “solid solution” of the agent in the polymer and would not consider that such a preparation would enable a “solution” to be formed on the addition of water. The skilled worker would also understand that there are practical difficulties with varying the compositions of solid solutions and these often only occur within narrow concentration ranges.

Our own co-pending international patent application PCT/GB03/03226 describes the formation of solid, porous beads comprising a three dimensional open-cell lattice of a water-soluble polymeric material. These are typically ‘templated’ materials formed by the removal of both water and a non-aqueous dispersed phase from an emulsion which has a polymer dissolved in the aqueous phase. The beads are formed by dropping the emulsion into a low temperature fluid such as liquid nitrogen, then freeze-drying the particles formed to remove the bulk of the aqueous phase and the dispersed phase. This leaves behind the polymer in the form of a porous structure. The beads dissolve rapidly in water and have the remarkable property that a water-insoluble component dissolved in the dispersed phase of the emulsion prior to freezing and drying can also be dispersed in water on solution of the polymer matrix of the beads. These materials do not have the formulation constraints of solid solutions, but as will be evident from the production process the beads have a relatively low density.

Our own co-pending applications GB 0501835 and GB 0613925 (filed 13 Jul. 2006) describe how powder materials which will form a nano-dispersion in water can be prepared, preferably by a spray-drying process. In the first of these applications the water insoluble materials are dissolved in the solvent-phase of a water/solvent emulsion. In the second, the water-insoluble materials are dissolved in a mixed solvent system and co-exist in the same phase as a water-soluble structuring agent. In both cases the liquid is dried above ambient temperature (above 20 Celsius), such as by spray drying, to produce powders of the structuring agent, as a carrier, with the water-insoluble materials dispersed therein. When these powders are placed in water they dissolve, forming a nano-dispersion of the water-insoluble material with particles typically below 500 nm and often below 300 nm. This scale is similar to that of virus particles, and the water-insoluble material behaves as though it were in solution. Spray dried powders can have a bulk density as low as 0.05 g/cm³.

In the present application the term ‘ambient temperature’ means 20 degrees Celsius and all percentages are percentages by weight unless otherwise specified.

While the nano-dispersion approach represents a significant improvement over grinding/milling and the solid solution approach, larger particle size powders have benefits over spray dried powders in terms of ease of handling, flow characteristics and bulk density.

### BRIEF DESCRIPTION OF THE INVENTION

We have now determined that improvements upon both the emulsion-based and the single-phase method can be used to produce bulk quantities of granular products comprising a dispersion of water-insoluble nano-particles in a water-soluble carrier. Surprisingly, this could be done using existing equipment which is broadly available. We have determined that a similar process exists for oil-insoluble actives.

Accordingly, a first aspect of the present invention provides a process for the production of a water-soluble composition comprising a water-insoluble active which comprises the steps of:
a) providing a liquid mixture comprising:
   i) a dissolved water-insoluble active,
   ii) a dissolved water-soluble carrier,
   iii) a solvent for each of the active and the carrier, and
b) spray-granulating the mixture to remove the, or each, solvent and obtain particles comprising a substantially water and solvent-free nano-dispersion of the water-insoluble active in the carrier said water-insoluble active being in nano-particles having a size range of 999-20 nm

A second aspect of the present invention provides a process for the production of a water-soluble composition comprising a water-insoluble active which comprises the steps of:
a) providing a substantially water and solvent-free powder comprising a water-insoluble active dispersed in a water soluble carrier, said water-insoluble active being in nano-particles having a size range of 999-20 nm, and,
b) spray-granulating the powder to obtain larger and denser particles

The spray-granulation process is known in the art and is that in which a solution of material is sprayed onto a fluidised bed of solids. Preferably the bed of solids is a bed comprising solids which are themselves particles which comprise a nano-dispersion of the water-insoluble active in the water-soluble carrier. If run as a batch process, the undersized grains from the previous batch can be used as the starting (“germ”) material. In a continuous process, the undersized grains can be continuously separated from the product and continuously fed back into the process as germ material.

Two processes occur during granulation and which one of them predominates is dependent on the particular operating conditions of the apparatus.

In one process, the spray liquid coats the fluidised germs, causing particles to adhere together, and is then dried, forming an aggregated structure often described as a “bunch-of-grapes”. In known spray-granulation processes the material being sprayed is often a binder in solution and any actives present are only present in the germs.

In a second process the spray coats the fluidised particles results in an “onion” or layered structure. “Onion-like” granulates typically are harder and more dense than “grape-like” aggregates. The formation of these layered structures is preferred in the method of the present invention and requires the material being sprayed to include the actives present. The germs for the spray-granulation process need not contain the actives present. However, the “grape”-type structures can be made with simpler apparatus.

Combinations of these two processes can be used, so as to produce mixed structures. For example a “grape-like structure” covered with layers.

In the spray-granulation process of the present invention at least one of the following conditions is satisfied:
a) the germs are a nano-dispersion of a water-insoluble active in a water soluble carrier, and/or,
b) the spray liquid forms such a nano-dispersion when drying on the surfaces of the germs.

Preferably, the bulk density of the spray-granulated material is in excess of 0.2 g/cm³. Preferred bulk densities are in the range 0.4-4 g/cm³, more preferably 0.4-1 g/cm³.

Spray granulation has the capability to generate relatively large particles, with sizes ranging from 20 microns up to 7 mm having been reported. Spray granulated materials are advantageous in that they are dust free, and for example can be round pellets. They show good flow behaviour and are therefore easy to dose. They have good dispersibility and solubility, a compact structure and low hygroscopicity.

Preferably the particles produced by the spray-granulation step have a weight average particle size greater than 30 micron, preferably greater than 100 microns, more preferably greater than 200 microns. Particularly preferred materials have a bulk density above 0.4 g/cm³ and a weight average particle size above 200 microns.

The preferred method of particle sizing for the nano-dispersed products of the present invention employs a dynamic light scattering instrument (Nano S, manufactured by Malvern Instruments UK). Specifically, the Malvern Instruments Nano S uses a red (633 nm) 4 mW Helium-Neon laser to illuminate a standard optical quality UV cuvette containing a suspension of material. The particle sizes quoted in this application are those obtained with that apparatus using the standard protocol. Particle sizes in solid products are the particle sizes inferred from the measurement of the particle size obtained by solution of the solid in water and measurement of the particle size

Preferably, the peak diameter (the so-called “Z-average”) of the water-insoluble active, when re-dissolved is below 800 nm. More preferably the peak diameter of the water-insoluble active is below 500 nm. In a particularly preferred embodiment of the invention the peak diameter of the water-insoluble active is below 200 nm.

It is believed that a relatively small particle size in the eventual nano-dispersion has significant advantages in improving the availability of the otherwise water-insoluble material. This is believed to be particularly advantageous where an improved bio-availability is sought, or, in similar applications where high local concentrations of the material are to be avoided. Moreover it is believed that nano-dispersions with a small particle size are more stable than those with a larger particle size.

Where particle sizes are given above 20 microns, this should be taken as the particle size range as measured by sieving, where more than 90% wt of the particles are in the specified range.

In the context of the present invention, “water insoluble” as applied to the active means that its solubility in water is less than 10 g/L. Preferably, the water insoluble active has solubility in water at ambient temperature (20 Celsius) less than 5 g/L preferably of less than 1 g/L, more preferably less than 200 mg/L, especially preferably less than 150 mg/L, even more preferably less than 100 mg/L. This solubility level provides the intended interpretation of what is meant by water-insoluble in the present specification.

Preferred carrier materials are selected from the group consisting of water-soluble inorganic materials, surfactants, polymers and mixtures thereof.

A further aspect of the present invention provides a process for preparing a composition comprising a water-insoluble active and a water-soluble carrier, which comprises the steps of:
a) forming an emulsion comprising:
   i) a solution of the active in a water-immiscible solvent for the same, and
   ii) an aqueous solution of the carrier, and,
b) spray-granulating the emulsion to remove water and the water-immiscible solvent to obtain a substantially water and solvent-free nano-dispersion of the active in the carrier

For convenience, this class of method is referred to herein as the “emulsion” method.

A further aspect of the present invention provides a process for preparing a composition comprising a water insoluble active and a water-soluble carrier which comprises the steps of:
a) providing a single phase mixture comprising:
   i) at least one non-aqueous solvent
   ii) optionally, water
   iii) a water-soluble carrier material soluble in the mixture of (i) and (ii) and
   iv) a water-insoluble active which is soluble in the mixture of (i) and (ii), and,
b) spray-granulating the solution to remove water and the water miscible solvent to obtain a substantially water and solvent-free nano-dispersion of the active in the carrier.

For convenience, this class of method is referred to herein as the “single-phase” method.

In the context of the present invention substantially solvent free means that the free solvent content of the product is less than 15% wt, preferably below 10% wt, more preferably below 5% wt and most preferably below 2% wt.

In the context of the present invention it is essential that both the carrier material and the active are essentially fully dissolved in their respective solvents prior to the drying step. It is not within the ambit of the present specification to teach the drying of slurries. For the avoidance of any doubt, it is therefore the case that the solids content of the emulsion or the mixture is such that over 90% wt, preferably over 95%, and more preferably over 98% of the soluble materials present is in solution prior to the drying step.

The ‘single phase’ method where both the active and the carrier material are dissolved in a phase comprising at least one non-aqueous solvent (and optional water) is preferred.

A further aspect of the present invention provides a solvent-free, granular product with a particle size in the range 20 micron to 10 mm and a bulk-density of greater than 0.4 g/cm3 comprising a water-soluble carrier material, the carrier material having dispersed therein a water-insoluble active, wherein on addition of water the carrier dissolves to form an aqueous dispersion of the active with a peak particle size of below 800 nm, preferably below 500 nm and more preferably below 200 nm.

Advantageously, the relatively coarse-grained high bulk density materials obtained by spray-granulation have a dense surface, a narrow grain size distribution and exhibit low abrasion.

While the process has been described above with particular reference to water-insoluble actives, the method can also be applied to water-soluble actives so as to form nano-particles of these actives in an oil-soluble matrix. For the purposes of the present invention “oil” is any water-immiscible solvent for the matrix in which the active is not normally soluble. In this “reverse” method the invention provides:

A process for the production of an oil-soluble composition comprising a water-soluble active which comprises the steps of:
a) providing a liquid mixture comprising:
   i) a dissolved water-soluble active,
   ii) a dissolved oil-soluble carrier,
   iii) a solvent for each of the active and the carrier, and
b) spray-granulating the mixture to remove the, or each, solvent and obtain particles comprising a substantially water and solvent-free nano-dispersion of the water-soluble active in the carrier said water-soluble active being in nano-particles having a size range of 999-20 nm

As with the spray-granulation of nano-dispersions of water-insoluble materials in a water-soluble carrier, the oil soluble carrier based materials can also be spray granulated.

Thus, a further aspect of the present invention provides a process for the production of an oil-soluble composition comprising a water-soluble active which comprises the steps of:
a) providing a substantially solvent-free powder comprising a water-soluble active dispersed in an oil-soluble carrier, said water-soluble active being in nano-particles having a size range of 999-20 nm, and,
b) spray-granulating the powder to obtain larger and denser particles

The products based on a water-soluble active can also be produced by the “emulsion” route.

Thus, a further aspect of the present invention provides a process for preparing a composition comprising a water-soluble active and a water-in soluble carrier, which comprises the steps of:
a) forming an emulsion comprising:
   i) a solution of the carrier in a water-immiscible solvent for the same, and
   ii) an aqueous solution of the active, and,
b) spray-granulating the emulsion to remove water and the water-immiscible solvent to obtain a substantially water and solvent-free nano-dispersion of the active in the carrier

The products based on a water-soluble active can also be produced by the “single-phase” route.

Thus, a further aspect of the present invention provides a process for preparing a composition comprising an water-soluble active and an oil-soluble carrier which comprises the steps of:
a) providing a single phase mixture comprising:
   i) at least one non-aqueous solvent
   ii) optionally, water
   iii) an oil-soluble carrier material soluble in the mixture of (i) and (ii) and
   iv) a water-soluble active which is soluble in the mixture of (i) and (ii), and,
b) spray-granulating the solution to remove water and the water miscible solvent to obtain a substantially water and solvent-free nano-dispersion of the active in the carrier.

Analogously with the case of water-insoluble actives, in the spray drying procedure at least one of the following conditions is satisfied:
a) the germs are a nano-dispersion of a water-soluble active in an oil-soluble carrier, and/or,
b) the spray liquid forms such a nano-dispersion when drying on the surfaces of the germs.

Analogous products are formed by the “reverse” process thus a yet further aspect of the invention provides solvent-free, granular product with a particle size in the range 20 microns to 10 mm and a bulk-density of greater than 0.4 g/cm3 comprising an oil-soluble carrier material, the carrier material having dispersed therein a water-soluble active, wherein on addition of oil the carrier dissolves to form an dispersion of the active with a peak particle size of below 800 nm, preferably below 500 nm and more preferably below 200 nm.

### DETAILED DESCRIPTION OF THE INVENTION

Various preferred features and embodiments of the present invention are described in further detail below. Generally, the invention will be described with reference to the production of water-soluble forms of oil-insoluble materials (for example, the oil-soluble and normally water-insoluble agrochemicals), however it will be apparent which process modifications are required for the production of oil-soluble forms of water-soluble materials (for example water-soluble salts such as sodium chloride).

### Actives

A wide range of useful water-insoluble actives are suitable for use in the method of the present invention either as single compounds of a mixtures of materials which are similar or dissimilar in activity.

These include cosmetic actives for example, antidandruff agents: for example: zinc pyrithione; skin lightening agents, for example 4-ethylresorcinol; skin conditioning agents: for example: cholesterol; hair conditioning agents: for example: quaternary ammonium compounds, protein hydrolysates, peptides, ceramides and hydrophobic conditioning oils for example hydrocarbon oils such as paraffin oils and/or mineral oils, fatty esters such as mono-, di-, and triglycerides, silicone oils such as polydimethylsiloxanes (e.g. dimethicone) and mixtures thereof.

They also include photo-active materials including dyes and pigments, also including fluorescing agents: for example: 2,5-bis(2-benzoxazolyl) thiophene for use on fabrics (such as cotton, nylon, polycotton or polyester) in laundry products; UV protecting agents: such as sunscreens for example: octyl methoxycinnamate (Parsol MCX), butyl methoxydibenzoylmethane (Parsol 1789) and benzophenone-3 (Uvinul M-40), and ferulic acid.

Further useful water-insoluble actives include thickening agents: for example: hydrophobically modified cellulose ethers such as modified hydroxyethyl-celluloses; bleach or bleach precursors: for example: 6-N-phthalimidoperoxyhexanoic acid (PAP) or photobleaching compounds; perfumes or flavourings or precursors thereto and antioxidants: for example: antioxiants based on hydroxytoluene such as Irganox™ or commercially available antioxidants such as the Trollox™ series.

Particularly preferred water-insoluble actives include pharmaceutically and otherwise biologically active compounds, such as biocides and agrochemicals. It is advantageous to be able to provide such materials as a water-dispersible granulate as this greatly reduces the risk and increases convenience when handling them. In particular a granulate reduces the risk of inhalation and inappropriate application of the product by wind-spreading for example.

The present invention is applicable to a broad range of water-insoluble pharmaceutically active agents, which can be granulated in a water-soluble carrier which, upon solution in water, releases a nano-dispersion of the pharmaceutically active agent. Many useful pharmaceutical agents have low solubility and these include, for example, sartans, statins, NSAIDS, antifungals, antiparasitics, vasodilators, CNS actives, antihypertensives, hormones, anticancer agents, sterols, analgesics, anaesthetics, antivirals, antiretrovirals, antihistamines, antibacterials, and antibiotics.

Preferred pharmaceutically active agents include water insoluble vitamins (such as vitamin E, retinol) and vitamin-like substances such as co-enzyme Q (ubiquinone).

The present invention is applicable to a broad range of water-insoluble biocides, which can be granulated in a water-soluble carrier which, upon solution in water, releases a nano-dispersion of the biocide. Preferred water-insoluble biocides for use in the present invention are antibacterials (for example chlorophenols including Triclosan), antifungals (for example organochlorines including Chlorothalonil and imidazoles such as Ketoconazole and Propiconazole) and/or herbicides (for example phenol-ureas including Isoproturon). The invention is also envisaged to be applicable to acaricides, algicides, insecticides, molluscicides and nematacides. In the context of the present invention the term biocide also incudes biostats. For example propiconazole is “fungistatic” rather than “fungicidal” as its mode of action involves inhibition of cell mitosis, rather than causing cell death.

Other agrochemicals include animal pesticides (for example rodenticides), plant growth regulators and fertilizers.

Specific water-insoluble agrochemical materials, with a solubility of less than or equal to 200 mg/L include: Gamma-cyhalothrin, Deltamethrin, Fluvalinate, Fenvalerate, Esfenvalerate, Flucycloxuron, Cyfluthrin, Metaflumizone, Clofentezine, Bifenthrin, Novaluron, Alpha-cypermethrin, Flufenoxuron, Lambda-cyhalothrin, Acequinocyl, Cypermethrin, Zeta-cypermethrin, Ethalfluralin, Teflubenzuron, Pyridaben, Cyflufenamid, Fenbutatin oxide, Tefluthrin, Chlorfluazuron, Acrinathrin, Etofenprox, Fenpyroximate, Hexaflumuron, Cyflumetofen, Flubendiamide, Bistrifluoron, Dimethomorph, Triflumuron, Azocyclotin, Silthiofam, Lufenuron, Picolinafen, Quinoxyfen, Diflufenican, Spirodiclofen, Benzobicyclon, Cinidon-ethyl, Diafenthiuron, Sulphur, Quinclorac, Benfluralin, Prothiofos, Etoxazole, Diflubenzuron, Pyraflufen, Cycloprothrin, Bifenox, Chlorethoxyfos, Hexythiazox, Amitraz, Fenazaquin, Carbosulfan, Famoxadone, Chlorfenapyr, Cyazofamid, Oxyfluorfen, Benzofenap, Spiromesifen, Fluazinam, Dinocap, Flumiclorac, Noviflumuron, Permethrin, Indoxacarb, Propargite, Pentoxazone, Trifluralin, Diflovidazin, Meptyldinocap, Paclobutrazol, Quizalofop, Pencycuron, Butralin, Endosulfan, Pendimethalin, Fenpropathrin, Fluacrypyrim, Oxadiargyl, Pyriproxyfen, Dithianon, Quintozene, Buprofezin, Metrafenone, Chlorthal, Lactofen, Oxadiazon, Trifloxystrobin, Propaquizafop, Zoxamide, Metamifop, Cyhalofop, Tolclofos-methyl, Diclomezine, Naproanilide, Bensultap, Dicofol, Folpet, Chlorothalonil, Tebufenozide, Fluthiacet, Milbemectin, Fenoxaprop, Tolylfluanid, Isoxaben, Proquinazid, Ziram, Fluazifop, Cyhexatin, Chinomethionat, Chlorantraniliprole, Chlorpyrifos, Tridemorph, Chromafenozide, Fluquinconazole, Abamectin, Dichlofluanid, Dithiopyr, Aclonifen, Phosalone, Pyridate, Phoxim, Haloxyfop, Oryzalin, Imibenconazole, Flumioxazin, Pyriftalid, Phenmedipham, Fludioxonil, Carpropamid, Isoxathion, Pyraclostrobin, Ethion, Kresoxim-methyl, Metiram, Benomyl, Bifenazate, Mepanipyrim, MCPA-thioethyl, Fluoxastrobin, Fentrazamide, Tebufenpyrad, Procymidone, Cafenstrole, Thiazopyr, Phthalide, Nitrothal-isopropyl, Lenacil, Flusulfamide, Fluopicolide, Picoxystrobin, Quizalofop-P-tefuryl, Beflubutamid, Methoxyfenozide, Vinclozolin, Pyribenzoxim, Bromobutide, Fenbuconazole, Fipronil, Bitertanol, Mefenacet, Clodinafop, Diniconazole, Triallate, Fenthion, Mandipropamid, Pyrazophos, Dimoxystrobin, Fenpropimorph, Terbufos, Azinphos-methyl, Boscalid, Ethaboxam, Esprocarb, Simazine, Captan, Profoxydim, Tralkoxydim, Isoxaflutole, Mancozeb, Diclosulam, Cyclosulfamuron, Terbuthylazine, Azoxystrobin, Imazosulfuron, Ipconazole, Desmedipham, Epoxiconazole, Penthiopyrad, Thifluzamide, Acibenzolar, Fenamidone, Fenoxycarb, Carbendazim, Flutolanil, Benfuracarb, Uniconazole, Lindane, Propazine, Propyzamide, Triforine, Fentin, Carbaryl, Triticonazole, Butafenacil, Bacillus thuringiensis, Propineb, Zineb, Triflumizole, Flurtamone, Thenylchlor, Pirimiphos-methyl, Phenthoate, Flamprop-M, Iprodione, Halofenozide, Mepronil, Cyprodinil, Bupirimate, Benthiavalicarb, Prosulfocarb, Anilofos, Fenarimol, Tecloftalam, Pyrazosulfuron, Dichlobenil, Difenoconazole, Phosmet, Thiram, Thiobencarb, Indanofan, Quinalphos, Iprovalicarb, Hexaconazole, Fenitrothion, Butachlor, Dimepiperate, Alanycarb, Spinetoram, Fenhexamid, Thiophanate, Thidiazuron, Quinoclamine, Carfentrazone, Terbutryn, Thiodicarb, Imazalil, Emamectin benzoate, Disulfoton, Nuarimol, Diethofencarb, Methiocarb, Profenofos, Benalaxyl, Spirotetramat, Benzoximate, Fenothiocarb, Thiabendazole, Metconazole, Prometryn, Pyraclofos, Diflumetorim, Norflurazon, Prochloraz, Atrazine, Triazophos, Fluorochloridone, Diuron, Tebuconazole, Bromuconazole, Flusilazole, Cyclanilide, Ethofumesate, Ethametsulfuron, Pretilachlor, Fomesafen, Phorate, Cycloxydim, Isoprothiolane, Parathion-methyl, Flufenacet, Edifenphos, Simeconazole, Methabenzthiazuron, Diazinon, Cinmethylin, Linuron, Bensulfuron, Primisulfuron, Sulfometuron, Triadimefon, Isoproturon, Fuberidazole, Triadimenol, Penconazole, Chlorotoluron, Napropamide, Orysastrobin, Oxpoconazole, Bromoxynil, Cyproconazole, Pyridaphenthion, Dodemorph, Fluometuron, Flucetosulfuron, Etridiazole, Pyrimethanil, Myclobutanil, Metominostrobin, Flutriafol, Carboxin, Malathion, Propiconazole, Tetraconazole, Mesotrione, Prohexadione, Maneb, Cloransulam, Thiacloprid, Metaldehyde, Pinoxaden, and Ametryn. It is envisaged that these materials may be formulated as described in the present specification.

### Water-Dispersible Product Form

The present invention provides a method for obtaining a water-dispersible form of an otherwise water-insoluble material. This is prepared by forming a not wholly aqueous intermediate emulsion or solution in which both a water-soluble carrier material and the water insoluble active are dissolved. On removal of solvents the insoluble active is left dispersed through the water-soluble carrier material. Suitable carrier materials are described in further detail below.

It is believed that the resulting dry materials are not encapsulates, as discrete micron sized bodies of the water-insoluble materials are not present in the dry product. Neither are the dry materials ‘dry emulsions’ as little or none of the volatile solvent comprising the ‘oil’ phase of the emulsion remains after the drying step. On addition of water to the dry product the initial emulsion is not reformed, as it would be with a ‘dry emulsion’. It is also believed that the compositions are not so-called solid solutions, as with the present invention the ratios of components present can be varied without loss of the benefits. Also from X-ray and DSC studies, it is believed that the compositions of the invention are not solid solutions, but comprise nano-scale, phase-separated mixtures.

Preferably, the compositions produced after the drying step will comprise the active and the carrier in a weight ratio of from 1:500 to 9:1 (as active:carrier), 1:9 to 9:1 being preferred.

By the method of the present invention the particle size of the active materials can be reduced to below 750 nm and may be reduced to around 15 nm. Preferred particle sizes are in the range 50-500 nm.

Where the active is a drug the water-dispersable product form may be administered by parenteral, oral, intraocular, topical and transdermal, rectal, or inhalable routes and may or may not need to be re-dispersed in an aqueous medium before administration.

### ‘Emulsion’ Preparation Method

In one preferred method according to the invention the solvent for the water-insoluble active is not miscible with water. On admixture with water it therefore can form an emulsion. This emulsion is used as the spray feedstock in the spray-granulator.

Preferably, the non-aqueous phase comprises from about 10% to about 95% v/v of the emulsion, more preferably from about 20% to about 68% v/v.

The emulsions are typically prepared under conditions which are well known to those skilled in the art, for example, by using a magnetic stirring bar, a homogeniser, or a rotational mechanical stirrer. The emulsions need not be particularly stable, provided that they do not undergo extensive phase separation prior to drying.

Homogenisation using a high-shear mixing device is a particularly preferred way to make an emulsion in which the aqueous phase is the continuous phase. A jet homogeniser gives particularly good results. It is believed that this avoidance of coarse emulsion and reduction of the droplet size of the dispersed phase of the emulsion, results in an improved dispersion of the ‘payload’ material in the dry product.

In a preferred method according to the invention a water-continuous emulsion is prepared with an average dispersed-phase droplet size (using the Malvern peak intensity) of between 50 nm and 5000 nm. We have found that an ‘Ultra-Turrax’ T25 type laboratory homogenizer (or equivalent) gives a suitable emulsion when operated for more than a minute at above 10,000 rpm.

There is a directional relation between the emulsion droplet size and the size of the particles of the ‘payload’ material, which can be detected after dispersion of the materials of the invention in aqueous solution. We have determined that an optional increase in the speed of homogenization for precursor emulsions can decrease final particle size after re-dissolution. For example, experiments have shown that the re-dissolved particle size can be reduced by nearly one half when the homogenization speed increased from 13,500 rpm to 21,500 rpm. The homogenization time is also believed to play a role in controlling re-dissolved particle size. The particle size again decreases with increase in the homogenization time, and the particle size distribution become broader at the same time.

Sonication is also a particularly preferred way of reducing the droplet size for emulsion systems. We have found that a Hert Systems Sonicator XL operated at level 10 for two minutes is suitable.

It is believed that ratios of components which decrease the relative concentration of the active to the solvents and/or the carrier give a smaller particle size.

### ‘Single Phase’ Preparation Method:

In an alternative method according to the present invention both the carrier and the active are soluble in a non-aqueous solvent or a mixture of such a solvent with water. Both here and elsewhere in the specification the non-aqueous solvent can be a mixture of non-aqueous solvents. It is this single phase mixture which is used as the feedstock for the spray granulator. This is a preferred method of carrying out the present invention.

In this case the feedstock of the drying step can be a single phase material in which both the water-soluble carrier and the water-insoluble active are dissolved. It is also possible for this feedstock to be an emulsion, provided that both the carrier and the active are dissolved in the same phase.

The ‘single-phase’ method is generally believed to give a better nano-dispersion with a smaller particle size than the emulsion method.

It is believed that ratios of components which decrease the relative concentration of the active to the solvents and/or the carrier give a smaller particle size.

In a further alternative method according to the present invention, either a single phase method or an emulsion method is used to produce a low density spray-dried powder. In a variant of this method the powder is obtained by freeze-drying the starting solution or emulsion. This low density powder is then spray-granulated with a binder solution, to produce a higher density granular product. The binder solution may or may not be an emulsion or solution as used in the “Single Phase” or “Emulsion” methods described above.

In a particularly preferred method according the present invention the spray granulator is initially started empty and is used as a spray drier to produce a relatively low density product. This product is not withdrawn from the drier but is kept in residence so that it forms the germs for a spray-granulation process as further emulsion or solution is sprayed onto the particles initially formed.

### Drying

Spray granulation is known to those versed in the art. In the case of the present invention some care must be taken due to the presence of a volatile non-aqueous solvent in the emulsion being granulated. In order to reduce the risk of explosion when a flammable solvent is being used, an inert gas, for example nitrogen, can be employed as the drying medium in a so-called closed spray-granulation system. The solvent can be recovered and re-used.

It is preferable that the process conditions should be

Inlet temperature: 40° C. to 250° C., more preferably 55° C. to 130° C.;

Outlet temperature: 20° C. to 250° C., more preferably 35° C. to 100° C.;

Feed concentration: 1-50% wt dissolved solids, more preferably 10-40% wt dissolved solids.

As noted above the spray-granulation process can be a continuous or a batch process.

Various modifications can be envisaged to the basic processes described above. The “germs” used may be of different composition than the material formed by drying of the spray liquid and or may be a mixture of materials. For example, they may comprise soluble or insoluble particles selected from glass beads, inorganic material, natural particles including clays, starch etc. Sequential or simultaneous spraying of spray liquids having different compositions, i.e. different actives and/or different carriers may be employed, for example final coatings of colourings or protective agents.

In one particularly advantageous embodiment of the invention the natural particles, used as ‘germs’ comprise living materials, including plant seeds, fungal spores other living matter.

As noted above, in the process of the present invention at least one of the following conditions is satisfied on at least one occasion:
a) the germs are a nano-dispersion of a water-insoluble active in a water soluble carrier, and/or,
b) the spray liquid forms such a nano-dispersion when drying on the surfaces of the germs.

Variants of the process include that in which a pre-formed powder containing a nano-dispersion of an active material (or a mixture of the same) is agglomerated with a spray liquid which may or may not be the same as that used to form the pre-formed powder and which may or may not itself form further nano-dispersed material. Examples of the spray liquid include solutions comprising polymers/binder, or emulsions and/or solutions which comprise a solvent having dissolved therein further water-insoluble actives.

Further variants include those processes in which the “germs” are materials are described above and free of nano-dispersed actives. In these variants the spray liquid (being an emulsion and/or solution) forms a nano-dispersion on drying.

It is possible to operate a process according to the present invention with an initially empty granulator and begin by spray-drying to form germs. Instead of allowing the product to exit the apparatus it is maintained in situ while the same or a different emulsion or suspension is used to granulate the product initially formed and build up larger and denser particles. Such a process can be operated in batch mode, or once started, can be operated in a continuous mode by removing particles which reach some pre-determined size.

In all these variants it is characteristic of the invention that at least at some stage particles comprising a nano-disperse water-insoluble material in a water-soluble carrier are being grown and densified by the spray-granulation process. It is a preferred feature of the process that the spray liquid comprises both a water-insoluble carrier and a water-insoluble active in solution in one or more solvents and it is a further preferred feature that the germs for the spray-granulation process also comprise a nano-dispersion of a water-insoluble active in a water-insoluble carrier.

### Carrier Material

As noted above, the carrier material for the nano-particles is water soluble, which includes the formation of structured aqueous phases as well as true solution of molecularly mono-disperse species. Carrier materials for many applications can be selected from suitable GRAS or FDA approved material. Suitable carriers include inorganic material, surfactant, a polymer or may be a mixture of two or more of these.

It is envisaged that other non-polymeric, organic, water-soluble materials such as sugars can be used as the carrier. However the carrier materials specifically mentioned herein are preferred.

Suitable carrier materials (referred to herein as ‘water soluble carrier materials’) include preferred water-soluble polymers, preferred water-soluble surfactants and preferred water-soluble inorganic materials.

### Preferred Polymeric Carrier Materials

Examples of suitable water-soluble polymeric carrier materials include:
(a) natural polymers (for example naturally occurring gums such as guar gum, alginate, locust bean gum or a polysaccharide such as dextran; Polymers including synthetic and natural polymers eg carbohydrates and proteins
(b) cellulose derivatives for example xanthan gum, xyloglucan, cellulose acetate, methylcellulose, methyl-ethylcellulose, hydroxy-ethylcellulose, hydroxy-ethylmethyl-cellulose, hydroxy-propylcellulose, hydroxy-propylmethylcellulose, hydroxy-propylbutylcellulose, ethylhydroxy-ethylcellulose, carboxy-methylcellulose and its salts (eg the sodium salt—SCMC), or carboxy-methylhydroxyethylcellulose and its salts (for example the sodium salt);
(c) homopolymers of or copolymers prepared from two or more monomers selected from: vinyl alcohol, acrylic acid, methacrylic acid, acrylamide, methacrylamide, acrylamide methylpropane sulphonates, aminoalkylacrylates, aminoalkyl-methacrylates, hydroxyethylacrylate, hydroxyethylmethylacrylate, vinyl pyrrolidone, vinyl imidazole, vinyl amines, vinyl pyridine, ethyleneglycol and other alkylene glycols, ethylene oxide and other alkylene oxides, ethyleneimine, styrenesulphonates, ethyleneglycolacrylates and ethyleneglycol methacrylate
(e) cyclodextrins, for example beta-cyclodextrin
(f) mixtures thereof.

When the polymeric material is a copolymer it may be a statistical copolymer (heretofore also known as a random copolymer), a block copolymer, a graft copolymer or a hyperbranched copolymer. Co-monomers other than those listed above may also be included in addition to those listed if their presence does not destroy the water soluble or water dispersible nature of the resulting polymeric material.

Examples of suitable and preferred homopolymers include poly-vinylalcohol, poly-acrylic acid, poly-methacrylic acid, poly-acrylamides (such as poly-N-isopropylacrylamide), poly-methacrylamide; poly-acrylamines, poly-methyl-acrylamines, (such as polydimethylaminoethylmethacrylate and poly-N-morpholinoethylmethacrylate), polyvinylpyrrolidone, poly-styrenesulphonate, polyvinylimidazole, polyvinylpyridine, poly-2-ethyl-oxazoline poly-ethyleneimine and ethoxylated derivatives thereof.

Polyethylene glycol (PEG), polyvinylpyrrolidone (PVP), poly(2-ethyl-2-oxazaline), polyvinyl alcohol (PVA) hydroxypropyl cellulose and hydroxypropyl-methyl cellulose (HPMC) and alginates are preferred polymeric carrier materials.

### Preferred Surfactant Carrier Materials

Where the carrier material is a surfactant, the surfactant may be non-ionic, anionic, cationic, amphoteric or zwitterionic.

Examples of suitable non-ionic surfactants include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™, alkyl polyglucosides; stearol ethoxylates; alkyl polyglycosides.

Examples of suitable anionic surfactants include alkylether sulfates; alkylether carboxylates; alkylbenzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates.

Examples of suitable cationic surfactants include fatty amine salts; fatty diamine salts; quaternary ammonium compounds; phosphonium surfactants; sulfonium surfactants; sulfonxonium surfactants.

Examples of suitable zwitterionic surfactants include N-alkyl derivatives of amino acids (such as glycine, betaine, aminopropionic acid); imidazoline surfactants; amine oxides; amidobetaines.

Mixtures of surfactants may be used. In such mixtures there may be individual components which are liquid, provided that the carrier material overall, is a solid.

Alkoxylated nonionic's (especially the PEG/PPG Pluronic™ materials), phenol-ethoxylates (especially TRITON™ materials), alkyl sulphonates (especially SDS), ester surfactants (preferably sorbitan esters of the Span™ and Tween™ types) and cationics (especially cetyltrimethylammonium bromide—CTAB) are particularly preferred as surfactant carrier materials.

### Preferred Inorganic Carrier Materials

The carrier material can also be an water-soluble inorganic material which is neither a surfactant nor a polymer. Simple organic salts have been found suitable, particularly in admixture with polymeric and/or surfactant carrier materials as described above. Suitable salts include carbonate, bicarbonates, halides, sulphates, nitrates and acetates, particularly soluble salts of sodium, potassium and magnesium. Preferred materials include, sodium carbonate, sodium bicarbonate and sodium sulphate. These materials have the advantage that they are cheap and physiologically acceptable. They are also relatively inert as well as compatible with many materials found in pharmaceutical products.

Mixtures of carrier materials are advantageous. Preferred mixtures include combinations of surfactants and polymers. Which include at least one of:
a) Polyethylene glycol (PEG), polyvinylpyrrolidone (PVP), hydroxypropyl cellulose and hydroxypropyl-methyl cellulose (HPMC), alginates and, at least one of;
b) Alkoxylated nonionic's (especially the PEG/PPG Pluronic™ materials), phenol-ethoxylates (especially TRITON™ materials), alkyl sulphonates (especially SDS), ester surfactants (preferably sorbitan esters of the Span™ and Tween™ types) and cationics (especially cetyltrimethylammonium bromide—CTAB)

The carrier material can also be a water-soluble small organic material which is neither a surfactant, a polymer nor an inorganic carrier material. Simple organic sugars have been found to be suitable, particularly in admixture with a polymeric and/or surfactant carrier material as described above. Suitable small organic materials include mannitol, polydextrose, xylitol and inulin etc.

### Non-Aqueous Solvent

The compositions of the invention comprise at least one volatile, non-aqueous solvent. This may either be miscible with the other solvents in the feedstock for the relevant drying step or, together with those solvents may form an emulsion.

In one alternative form of the invention a single, non-aqueous solvent is employed in which can form a single phase with water in the presence of the active and the carrier. Preferred solvents for these embodiments are polar, protic or aprotic solvents. Generally preferred solvents have a dipole moment greater than 1 and a dielectric constant greater than 4.5.

Particularly preferred solvents are selected from the group consisting of haloforms (preferably dichloromethane, chloroform), lower (C1-C10) alcohols (preferably methanol, ethanol, isopropanol, isobutanol), organic acids (preferably formic acid, acetic acid), amides (preferably formamide, N,N-dimethylformamide), nitriles (preferably aceto-nitrile), esters (preferably ethyl acetate) aldehydes and ketones (preferably methyl ethyl ketone, acetone), and other water miscible species comprising hetroatom bond with a suitably large dipole (preferably tetrahydrofuran, dialkylsulphoxide).

Haloforms, lower alcohols, ketones and dialkylsulphoxides are the most preferred solvents.

In another alternative form of the invention the non-aqueous solvent is not miscible with water and forms an emulsion.

The non-aqueous phase of the emulsion is preferably selected from one or more from the following group of volatile organic solvents:
alkanes, preferably heptane, n-hexane, isooctane, dodecane, decane;
cyclic hydrocarbons, preferably toluene, xylene, cyclohexane;
halogenated alkanes, preferably dichloromethane, dichoroethane, trichloromethane (chloroform), fluoro-trichloromethane and tetrachloroethane;
esters preferably ethyl acetate;
ketones preferably 2-butanone;
ethers preferably diethyl ether;
volatile cyclic silicones preferably either linear or cyclomethicones containing from 4 to 6 silicon units. Suitable examples include DC245 and DC345, both of which are available from Dow Corning Inc.

Preferred solvents include dichloromethane, chloroform, ethanol, acetone and dimethyl sulphoxide.

Preferred non-aqueous solvents, whether miscible or not have a boiling point of less than 150 Celsius and, more preferably, have a boiling point of less than 100 Celsius, so as to facilitate drying, particularly spray-granulation under practical conditions and without use of specialised equipment. Preferably they are non-flammable, or have a flash point above the temperatures encountered in the method of the invention.

Preferably, the non-aqueous solvent comprises from about 10% to about 95% v/v of any emulsion formed, more preferably from about 20% to about 80% v/v. In the single phase method the level of solvent is preferably 20-100% v/v.

Particularly preferred solvents are alcohols, particularly ethanol and halogenated solvents, more preferably chlorine-containing solvents, most preferably solvents selected from (di- or tri-chloromethane).

### Optional Co-Surfactant

In addition to the non-aqueous solvent an optional co-surfactant may be employed in the composition prior to the drying step.

We have determined that the addition of a relatively small quantity of a volatile cosurfactant reduced the particle diameter of the material produced. This can have a significant impact on particle volume. For example, reduction from 297 nm to 252 nm corresponds to a particle size reduction of approximately 40%. Thus, the addition of a small quantity of co-surfactant offers a simple and inexpensive method for reducing the particle size of materials according to the present invention without changing the final product formulation.

Preferred co-surfactants are short chain alcohols or amine with a boiling point of <220° C.

Preferred co-surfactants are linear alcohols. Preferred co-surfactants are primary alcohols and amines. Particularly preferred co-surfactants are selected from the group consisting of the 3-6 carbon alcohols. Suitable alcohol co-surfactants include n-propanol, n-butanol, n-pentanol, n-hexanol, hexylamine and mixtures thereof.

Preferably the co-surfactant is present in a quantity (by volume) less than the solvent preferably the volume ratio between the solvent and the co-surfactant falls in the range 100:40 to 100:2, more preferably 100:30 to 100:5.

### Preferred Spray-Granulator Feedstocks

Typical spray-granulator feedstocks comprise:
a) a surfactant,
b) at least non-aqueous solvent,
c) more than 0.1% of at least one water-insoluble active dissolved in the feedstock,
d) a polymer, and,
e) optional water

Preferred spray-granulator feedstocks comprise:
a) at least one non-aqueous solvent selected from dichloromethane, chloroform, ethanol, ethyl acetate, acetone, and mixtures thereof,
b) a surfactant selected from PEG co-polymer nonionic's (especially the PEG/PPG Pluronic™ materials), alkyl sulphonates (especially SDS), ester surfactants (preferably sorbitan esters of the Span™ and Tween™ types) and cationics (especially cetyltrimethylammonium bromide—CTAB) and mixtures thereof,
c) more than 0.1% of at least one water-insoluble active,
d) a polymer selected from Polyethylene glycol (PEG), Polyvinyl alcohol (PVA), polyvinyl-pyrrolidone (PVP), hydroxypropyl cellulose and hydroxypropyl-methyl cellulose (HPMC), alginates and mixtures thereof, and
e) optionally water.

The drying feed-stocks used in the present invention are either emulsions or solutions which preferably do not contain any solid matter and in particular preferably do not contain any undissolved active.

It is particularly preferable that the level of the active in the composition should be such that the loading in the dried composition is below 40% wt, and more preferably below 30% wt. Such compositions have the advantages of a small particle size and high effectiveness as discussed above.

### Water-Dispersed Form

On admixture of the water-soluble carrier material with water, the carrier dissolves and the water-insoluble active is dispersed through the water in sufficiently fine form that it behaves like a soluble material in many respects. The particle size of the water-insoluble materials in the dry product is preferably such that, on solution in water the water-insoluble materials have a particle size of less than 1 micron as determined by the Malvern method described herein. It is believed that there is no significant reduction of particle size for the active on dispersion of the solid form in water.

By applying the present invention significant levels of ‘water-insoluble’ materials can be brought into a state which is largely equivalent to true solution. When the dry product is dissolved in water it is often possible to achieve optically clear solutions comprising more than 0.1%, preferably more than 0.5% and more preferably more than 1% of the water-insoluble material.

In order that the present invention may be further understood and carried forth into practice it is further described below with reference to non-limiting examples.

### EXAMPLES

### Example 1

### Preparation of Coenzyme Q10 in a Water Dispersible Form

Granulation carried out on Glatt type GPCG3.1 fluidized bed granulation apparatus

### Formulation

| | | |
|---|---|---|
| Material | Weight | % |

| | | | | |
|---|---|---|---|---|
| Coenzyme Q10 | 375 | g | 15 | |
| PVP K30 | 1250 | g | 50 | |
| Brij 58 | 875 | g | 35 | |
| | | | | Solids = 37% |
| Ethyl Acetate | 4.25 | L | | |
| Ethanol | 2.50 | L | | |

### Trial Conditions

| Inlet Temp. | Outlet Temp. | Product Temp. | Residual | Spray |
|---|---|---|---|---|
| ° C. | ° C. | ° C. | Moisture* | Pressure |
| 58-60 | 37-39 | 37-40 | 1.12 | 1.5 bar |

### Moisture by Loss on Drying (Ohaus Balance—Model MB45)

### Sieve Analysis

Sieve Analysis “Retsch”, micron, %

| <63 | >63 | >100 | >200 | >355 | >500 | >800 | >1000 |
|---|---|---|---|---|---|---|---|
| 2.2 | 1.9 | 13.2 | 59.1 | 22.5 | 1.1 | 0.0 | 0.0 |

### Particle Sizing

Dissolution in water of the spray-granulated product at 1 mg/ml produced a dispersion of the water insoluble active having a Z—average (as measured with the Malvern Instruments Nano S) of 187 nm.

### Example 2

### Preparation of Oil Red in a Water Dispersible Form

Agglomeration of a spray dried oil red formulation in ‘Procept’ 4M8 bench-top fluid bed.

### Formulation

10% Sudan Red Formulation

| Sudan Red (g) | Pluronic F127 (g) | K30 PVP (g) | Solvent - DCM |
|---|---|---|---|
| 5.0 | 10.0 | 35.0 | 500 ml |

DCM = Dichloromethane
PVP = Polyvinylpyrrolidone

The formulation given above was spray-dried. The powder thus produced was then used as seeds which were placed into the spray granulator. The same solution used to spray dry the seeds was sprayed onto the seeds in a ‘top-spray’ configuration in order to form the granulates

### Trial Conditions

| Inlet Temp. ° C. | Outlet Temp. ° C. | Product Temp. ° C. |
|---|---|---|
| 57-67 | 38-42 | 40-52 |

### Particle Sizing

Dissolution in water of the spray-granulated product at 1 mg/ml produced a dispersion of the water insoluble active having a Z—average (as measured with the Malvern Instruments Nano S) of 100 nm.

### Example 3

### Preparation of Sodium Chloride in an Oil-Dispersible Form

10 g of NaCl together with 20 g of Pluronic F68 and 70 g Polyvinylpyrrolidone k30 was dissolved in 1200 mL of 50:50 distilled water/ethanol.

This was spray granulated in a Lodige/DMR WFP-mini Fluidbed laboratory spray granulator. The spray configuration was bottom spray and the inlet/outlet temps were 100 C/40-48 C. The product temperature was 46 C. The feedstock flow rate was 5 ml/min. Drying gas was set at 25 m3/hr. No seeds/germs were loaded in the granulator to start the granulation. The feedstock was sprayed until ‘self-seeding’ occurred. It was found that the granule size could be grown to a larger diameter by increasing the duration of spray granulation (sizes of up to ˜5 mm were achieved).

When the spray granulated materials were dissolved in chloroform the following particle sizes for the “dissolved” NaCl were measured (using the Malvern) for the size fractions noted.

Spray granulated balls of <1 mm diameter: 232 nm (Z-average)

Spray granulated balls of 1-2 mm diameter: 255 nm (Z-average)

Spray granulated balls of 2-3 mm diameter: 275 nm (Z-average).

1. A process for the production of a water-soluble composition comprising a water-insoluble active which comprises the steps of:
a) providing a liquid mixture comprising:
i) a dissolved water-insoluble active,
ii) a dissolved water-soluble carrier, and
iii) a solvent for each of the active and the carrier; and
b) spray-granulating the mixture to remove the, or each, solvent and obtain a substantially water and solvent-free nano-dispersion of the water-insoluble active in the carrier, said water-insoluble active being in nano-particles having a size range of 20-999 nm.

2. A process for the production of a water-soluble composition comprising a water-insoluble active which comprises the steps of:
a) providing a substantially solvent-free powder comprising a water-insoluble active dispersed in a water soluble carrier, said water-insoluble active being in nano-particles having a size range of 20-999 nm, and
b) spray-granulating the powder to obtain larger and denser particles.

3. A process according to claim 1 which comprises the steps of:
a) providing an emulsion comprising:
i) a solution of the active agent in a water-immiscible solvent for the same, and
ii) an aqueous solution of the carrier; and
b) spray-granulating the emulsion to remove water and the water-immiscible solvent to obtain a granular, substantially water and solvent-free nano-dispersion of the active in the carrier.

4. A process according to claim 1 which comprises the steps of:
a) providing a single phase mixture comprising:
i) at least one non-aqueous solvent,
ii) optionally, water,
iii) a water-soluble carrier material soluble in the mixture of (i) and (ii), and
iv) a water-insoluble active which is soluble in the mixture of (i) and (ii); and
b) spray-granulating the solution to remove water and the water miscible solvent to obtain a granular, substantially water and solvent-free nano-dispersion of the active in the carrier.

5. A process according to claim 1 wherein the spray-granulation process is conducted at an inlet temperature 55° C. to 130° C. and an outlet temperature of from 35° C. to 100° C., and wherein the outlet temperature is lower than the inlet temperature.

6. A process according to claim 1 in which the carrier material includes a polymer, a surfactant, or a combination thereof.

7. A solvent-free, granular product produced by the method of claim 1.

8. A solvent-free, granular product with a particle size in the range of 20 microns to 10 mm and a bulk-density of greater than 0.4 g/cm³ comprising a water-soluble carrier material, the carrier material having dispersed therein a water-insoluble active, wherein on addition of water the carrier dissolves to form an aqueous dispersion of the active with a peak particle size of below 800 nm.

9. A process for the production of an oil-soluble composition comprising a water-soluble active which comprises the steps of:
a) providing a liquid mixture comprising:
i) a dissolved water-soluble active,
ii) a dissolved oil-soluble carrier, and
iii) a solvent for each of the active and the carrier; and
b) spray-granulating the mixture to remove the, or each, solvent and obtain particles comprising a substantially water and solvent-free nano-dispersion of the water-soluble active in the carrier said water-soluble active being in nano-particles having a size range of 20-999 nm.

10. A process for the production of an oil-soluble composition comprising a water-soluble active which comprises the steps of:
a) providing a substantially solvent-free powder comprising a water-soluble active dispersed in an oil-soluble carrier, said water-soluble active being in nano-particles having a size range of 20-999 nm, and
b) spray-granulating the powder to obtain larger and denser particles.

11. A process for preparing a composition comprising a water-soluble active and a water-insoluble carrier, which comprises the steps of:
a) forming an emulsion comprising:
i) a solution of the carrier in a water-immiscible solvent for the same, and
ii) an aqueous solution of the active; and
b) spray-granulating the emulsion to remove water and the water-immiscible solvent to obtain a substantially water and solvent-free nano-dispersion of the active in the carrier.

12. A process for preparing a composition comprising an water-soluble active and an oil-soluble carrier which comprises the steps of:
a) providing a single phase mixture comprising:
i) at least one non-aqueous solvent,
ii) optionally, water,
iii) an oil-soluble carrier material soluble in the mixture of (i) and (ii), and
iv) a water-soluble active which is soluble in the mixture of (i) and (ii); and
b) spray-granulating the solution to remove water and the water miscible solvent to obtain a substantially water and solvent-free nano-dispersion of the active in the carrier.

13. A solvent-free, granular product with a particle size in the range of 20 microns to 10 mm and a bulk-density of greater than 0.4 g/cm³ comprising an oil-soluble carrier material, the carrier material having dispersed therein a water-soluble active, wherein on addition of oil the carrier dissolves to form an dispersion of the active with a peak particle size of below 800 nm.

14. The product of claim 8, wherein the aqueous dispersion of the active has a peak particle size of below 500 nm.

15. The product of claim 8, wherein the aqueous dispersion of the active has a peak particle size of below 200 nm.

16. The product of claim 13, wherein the aqueous dispersion of the active has a peak particle size of below 500 nm.

17. The product of claim 13, wherein the aqueous dispersion of the active has a peak particle size of below 200 nm.
